(19) United States
(12) Patent Application Publication
(10) Pub. No.: US 2011/0130813 A1
(43) Pub. Date: Jun. 2, 2011

(54) THIN FILM ENERGY FABRIC FOR SELF-REGULATING HEATED WOUND DRESSINGS

(75) Inventor: Wylie Moreshead, Bainbridge Island, WA (US)

(73) Assignee: Kinaptic, LLC, Evergreen, CO (US)

(21) Appl. No.: 12/962,568

(22) Filed: Dec. 7, 2010

Related U.S. Application Data
(63) Continuation-in-part of application No. 11/972,577, filed on Jan. 10, 2008.
Application No. 11/972,577 is a continuation-in-part of application No. 11/439,572, filed on May 23, 2006, now Pat. No. 7,494,945.
Application No. 11/439,572 is a continuation-in-part of application No. 12/390,309, filed on Feb. 20, 2009.
Application No. 12/390,309 is a continuation of application No. 11/439,572, filed on May 23, 2006, now Pat. No. 7,494,945.
(60) Provisional application No. 60/684,890, filed on May 26, 2005.

(51) Int. Cl. A61F 7/08

(52) U.S. Cl. 607/112

(57) ABSTRACT

The Self-Regulating Heated Wound Dressing includes an energy storage section adapted to store electrical energy; an energy release section coupled to the energy storage section and configured to receive electrical energy from the energy storage section and to utilize the electrical energy in the generation of a thermal energy used to self-regulate the temperature of a heated wound dressing; and an energy recharge section, coupled to the energy storage section, adapted to receive or collect energy and convert the received or collected energy to electrical energy either for storage by the energy storage section or for use by the energy release section or simultaneous storage in the energy storage section and immediate use by the energy release section.

### CROSS-REFERENCE TO RELATED APPLICATIONS

This Application is a Continuation-In-Part of U.S. patent application Ser. No. 11/972,577 filed on Jan. 10, 2008, which is a Continuation-In-Part of U.S. patent application Ser. No. 11/439,572 filed on May 23, 2006, now U.S. Pat. No. 7,494,945 B2 issued Feb. 24, 2009, which claims the benefit of U.S. Provisional Patent Application No. 60/684,890 filed on May 26, 2005. This Application also is a Continuation-In-Part of U.S. patent application Ser. No. 12/390,209 filed on Feb. 20, 2009, which is a Continuation-In-Part of U.S. patent application Ser. No. 11/439,572 filed on May 23, 2006, now U.S. Pat. No. 7,494,945 B2 issued Feb. 24, 2009, which claims the benefit of U.S. Provisional Patent Application No. 60/684,890 filed on May 26, 2005. This application also is related to an application titled “Thin Film Energy Fabric With Energy Transmission/Reception Layer” and filed on the same date hereof; and to an application titled “Thin Film Energy Fabric With Light Generation Layer” and filed on the same date hereof; and to an application titled “Thin Film Energy Fabric With Self-Regulating Heat Generation Layer” and filed on the same date hereof. The above-referenced patent applications and patent are incorporated herein by reference in their entirety.

### FIELD OF THE INVENTION

The present Thin Self-Regulating Heated Wound Dressing is directed to thin, flexible material and, more particularly, to a flexible fabric having electrical energy storage, electrical energy release, and electrical energy transmission/reception capabilities integrally formed therewith for use in a heated wound dressing application.

### BACKGROUND OF THE INVENTION

A traditional problem with the application of heat to a wound is the thermal cycling of heated bandages, where the initial temperature of the bandage is above the desired temperature and the thermal output rapidly diminishes to a level below that desired for the selected use, thereby quickly reducing the efficacy of the heated bandage. The frequent replacement of the heated bandage can be damaging to the healing process and is costly in terms of materials and staff time required to manage the heat application process using the heated bandages.

Presently, there are materials that incorporate energy releases in the form of light or heat and are powered by some external, rigid power source. However, there is not a single fabric available to the engineer or designer that has the electrical energy storage aspect directly integrated into it and is still thin, flexible, and can be manufactured into a product with the same ease as conventional fabrics. Hence, there is a need in this day and age for such a fabric that also has all the normal characteristics of a modern engineered fabric, such as waterproof, breathability, moisture wickability, stretch, color, and texture choices. So far, no fabric has emerged with all these characteristics.

### BRIEF SUMMARY OF THE INVENTION

The Thin Film Energy Fabric For Self-Regulating Heated Wound Dressings (termed “Self-Regulating Heated Wound Dressing” herein) has all the characteristics of a modern engineered fabric, such as water resistance, waterproof, moisture wickability, breathability, stretch, and color and texture choices, along with the ability to store electrical energy and release it to provide a use of the stored electrical energy. In addition, the Self-Regulating Heated Wound Dressing can include a section that takes energy from its surroundings, converts it to electrical energy, and stores it inside the Self-Regulating Heated Wound Dressing for later use. This energy storage and release capability is used in the context of a heated wound dressing (also termed “bandage”) to be applied to a surface to stimulate healing of a wound or treatment of the area for stimulating circulation for pain relief, delivery of medicines, cosmetic treatments, and the like.

In particular, the Self-Regulating Heated Wound Dressing includes an energy storage section adapted to store electrical energy; an energy release section coupled to the energy storage section and configured to receive electrical energy from the energy storage section and to utilize the electrical energy in the generation of a thermal energy used to self-regulate the temperature of a heated wound dressing; and an energy recharge section, coupled to the energy storage section, adapted to receive or collect energy and convert the received or collected energy to electrical energy either for storage by the energy storage section or for use by the energy release section or simultaneous storage in the energy storage section and immediate use by the energy release section.

The Self-Regulating Heated Wound Dressing provides a predetermined thermal output to maintain a substantially constant temperature at the wound site for an extended period of time. Outputs also could be provided to include feedback on the wound condition, such as: moisture level, PH, oxygen level, etc. These outputs could be read in several different ways: possibly something as simple as a color change in the bandage signifying wound health or whether the dressing needs to be changed. The output could be as complex as a connector (for example, a mini-USB) where a doctor could connect an instrument and read back wound condition without having to remove the dressing. The dressing could also have its own readout (for example, light emission) or it could be transmitted wirelessly. The Self-Regulating Heated Wound Dressing is self-contained to enable the patient to be ambulatory and also is wirelessly rechargeable to provide the capability for producing a constant thermal output over an extended period of time without having to remove the dressing. The heated wound dressing can be coupled with an absorbent bandage fabric to interface between the wound surface and the Self-Regulating Heated Wound Dressing. In addition, the bandage fabric can be impregnated with therapeutic materials, such as medications, including thermally activated medications.

The Self-Regulating Heated Wound Dressing can include optional treatment and sealing, and optional protective and decorative sections. It should be noted that these various sections can be arranged coplanar or layered, as long as the sections are continually connected or enveloped together. In addition, the fabric may include one or more properties of semi-flexibility or flexibility, water resistance or waterproof, and formed as a thin, sheet-like material or a thin woven fabric. The Self-Regulating Heated Wound Dressing can be formed from strips of material having the characteristics described above and that are woven together to provide a thin, flexible material that can be utilized in conjunction with a conventional wound dressing or a specialized fabric panel.

### BRIEF DESCRIPTION OF THE DRAWINGS

The foregoing and other features and advantages of the present Self-Regulating Heated Wound Dressing will be more readily appreciated as the same become better understood from the following detailed description when taken in conjunction with the accompanying drawings, wherein:

FIG. 1 is an isometric illustration of the present Self-Regulating Heated Wound Dressing;

FIG. 2 is an isometric illustration of another embodiment of the present Self-Regulating Heated Wound Dressing;

FIG. 3 is an isometric illustration of a further embodiment of the present Self-Regulating Heated Wound Dressing;

FIG. 4 is an isometric illustration of yet another embodiment of the present Self-Regulating Heated Wound Dressing showing energy flow into and out of the fabric;

FIG. 5 illustrates embedded electronic components in film substrates;

FIGS. 6 and 7 illustrate two batten-forming adhesive patterns;

FIG. 8 illustrates the use of registration points in assembling components of energy textile panels;

FIGS. 9A-9C illustrate typical heated wound bandages using the Self-Regulating Heated Wound Dressing;

FIG. 10 illustrates a typical subcutaneous wound and the initial stages of the wound healing process;

FIG. 11 illustrates a typical subcutaneous wound and the various biological reactions involved in wound healing; and

FIG. 12 illustrates a typical wireless apparatus for the transfer of energy into and out of the Self-Regulating Heated Wound Dressing.

### DETAILED DESCRIPTION OF THE INVENTION

The present Self-Regulating Heated Wound Dressing consists of a number of components which can be used to apply a predetermined thermal output to the site to which the Self-Regulating Heated Wound Dressing is applied. In a general sense, the Self-Regulating Heated Wound Dressing includes a thermal generation material and an associated bandage material.

The bandage material in common terminology consists of some material that is designed to be cooperatively operating with the thermal generation material to produce the desired effect, typically: fluid absorbing, and/or cushioning, and/or product delivering, and/or insulating, and/or thermally dispersive, and the like. Thus, a bandage is a piece of material used either to support a medical device such as a dressing or splint, or on its own to provide support to the body. Bandages are available in a wide range of types, from generic cloth strips, to specialized-shaped bandages designed for a specific limb or part of the body, although bandages can often be improvised as the situation demands, using clothing, blankets, or other material. In common speech, the word “bandage” often is used to mean a dressing, which is used directly on a wound, whereas a bandage technically is only used to support a dressing and not be placed directly on a wound.

A dressing is an adjunct used by a person for application to a wound to promote healing and/or prevent further harm. A dressing is designed to be in direct contact with the wound, which makes it different from a bandage, which primarily is used to hold a dressing in place. Some organizations classify them as the same thing (for example, the British Pharmacopoeia), and the terms are used interchangeably by some people.

In a medical application, as described below, the Self-Regulating Heated Wound Dressing includes a thermal generation material and an associated bandage material. It functions to stimulate blood circulation to the site to facilitate healing of a wound; or it can be used to deliver medications (also termed “product” herein) to the site, with the increased blood flow increasing absorption of the medicines through the skin. The same absorption effect can be used for cosmetic or therapeutic purposes to deliver products associated with these applications to the site. Other possible components of the Self-Regulating Heated Wound Dressing are described below. In order to simplify the following description, the example used herein is that of a wound dressing applied to a wound site (which can be any locus desired in other applications).

### Architecture of the Self-Regulating Heated Wound Dressing

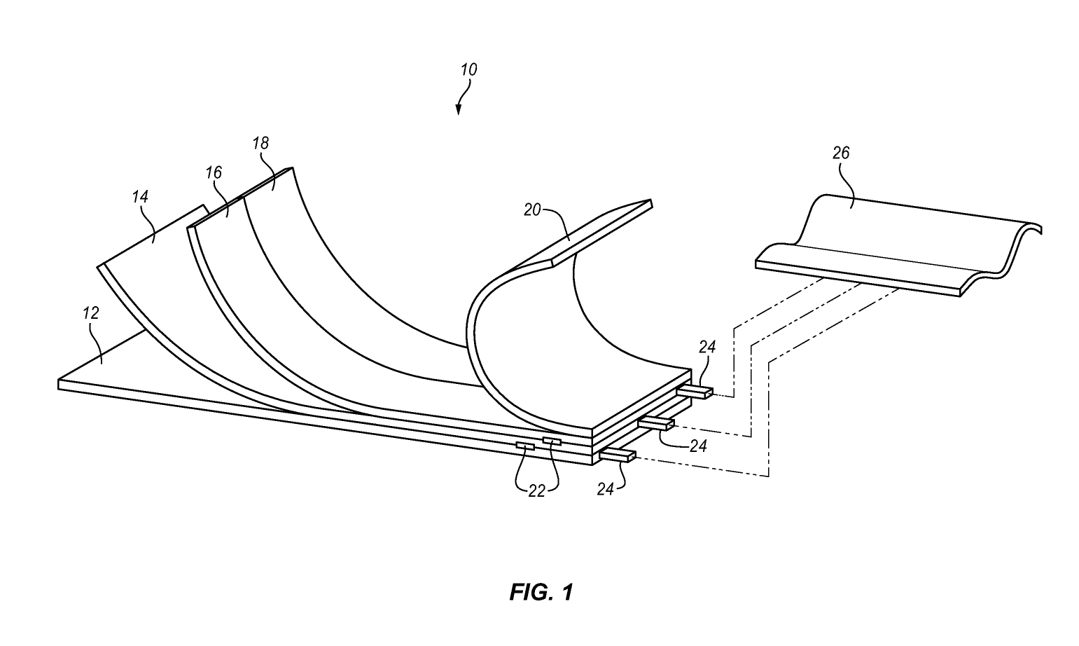

FIG. 1 illustrates the flexible sheet form of the finished Self-Regulating Heated Wound Dressing 10 that includes an energy release section 12 and an energy storage section 14. An optional charge section 16 or recharge section 18 or combination thereof is shown along with an optional protective section 20 that can also be a decorative section or a bandage section. These sections can be manufactured separately and then laminated together, or each section can be directly deposited on the one beneath it, or a combination of both techniques can be employed to produce the final Self-Regulating Heated Wound Dressing 10. These sections can be arranged in any order including coplanar arrangements, layers, planes, and other stacking arrangements; and there can be multiple instances of each section in the final Self-Regulating Heated Wound Dressing 10.

The sections also can have different embodiments on the same plane. For instance, a section of the charge or recharge plane 16, 18 can use photovoltaics while another section can use piezoelectrics, or a section of the energy release plane can produce light while another section can produce heat. Similarly, one section of the plane can produce light while another section on the same plane can use photovoltaics to recharge the energy storage section. Some sections must be connected electrically to some of the other sections. This can be done with the contact occurring at certain points 22 directly between the sections or with the contact occurring though leads 24 that connect via a Printed Circuit Board 26 which is either integrated into the Self-Regulating Heated Wound Dressing 10 or located external to the Self-Regulating Heated Wound Dressing 10, thus providing operator input, monitoring, and control capabilities. Although not required, this Printed Circuit Board 26 can be built on a flexible substrate as can the leads 24; and the Printed Circuit Board 26 can simultaneously control multiple separate Self-Regulating Heated Wound Dressing instances. Briefly, controls such as fixed and variable resistance, capacitance, inductance, and combinations of the foregoing, as well as software and firmware embodied in corresponding hardware, can be implemented to regulate voltage and current, phase relationships, timing, and other known variables that ultimately affect the output. Regulation can be user controlled or automatic or a combination of both.

The leads 24 that connect the sections can, but do not have to, be connected to the Printed Circuit Board 26. All lead connections should be sealed at the point of contact to provide complete electrical insulation. The flexible Printed Circuit Board 26, which contains circuits, components, switches and sensors, also can be integrated directly into the final fabric as another section, coplanar or layered, and so can the leads.

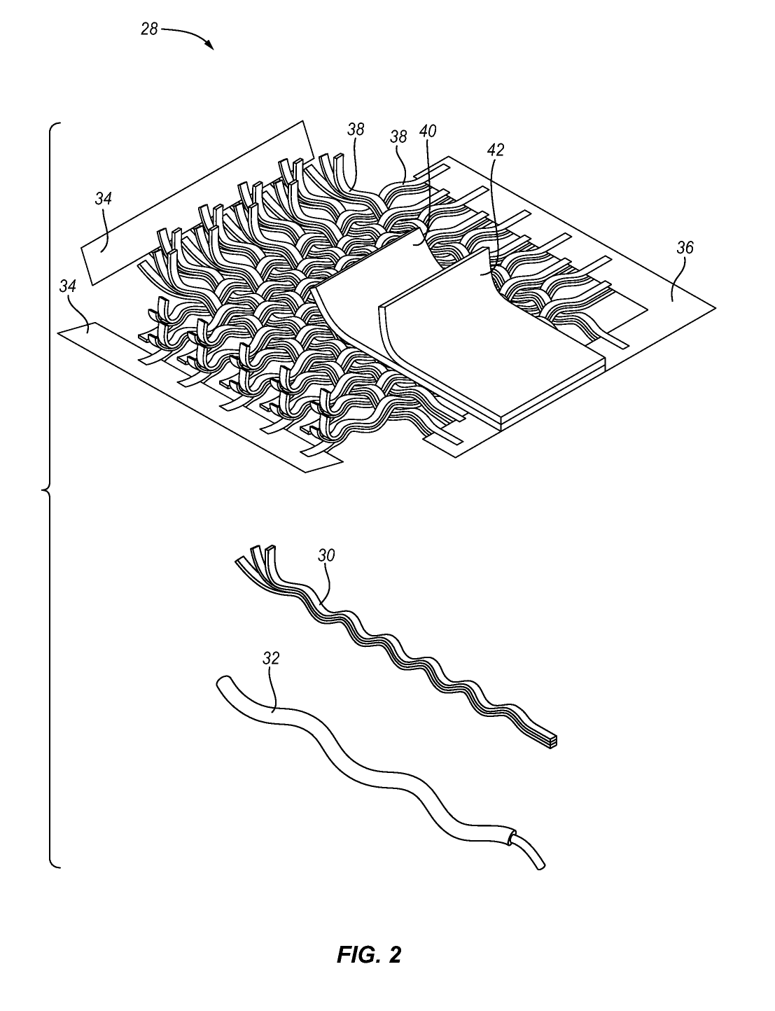

FIG. 2 illustrates the highly flexible woven form of a finished energy fabric 28 that includes woven strips 30 where each individual strip contains an energy release section, an energy storage section, and an optional charge/recharge section. The strips 30 would not necessarily need to be constructed with rectangular sections; they can also be constructed with coaxial sections 32. The strips 30 can be, but would not have to all be, electrically connected at the edge 34 of the fabric 28 with similar contacts 36 on the warp and weft of the weave being isolated at the same potential as applicable for the circuit to function. All of the strips 30 do not necessarily have to have the same characteristics. For instance, strips with different energy release embodiments can be woven into the same piece of fabric as shown at 38.

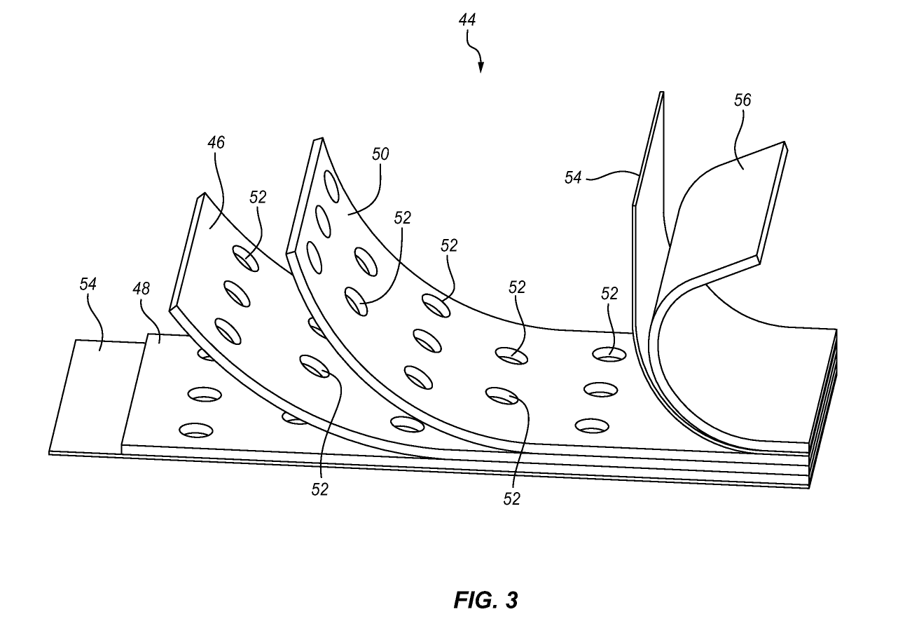

FIG. 3 illustrates a highly flexible sheet 44 consisting of an energy storage section 46, an energy release section 48, and an optional charge or recharge section 50, all patterned with openings 52 to impart traits such as breathability and flexibility to the final fabric. These openings or holes 52 in the fabric 44 can be deposited in a pattern for each section, with the sections then laminated together such that the patterns line up to provide an opening through the fabric covered only by a treatment or sealing enveloping section 54, and possibly a decorative or protective section 56; or the fabric 44 can have holes 52 cut into it after lamination but before the application of the treatment or sealing section 54 or the decorative or protective section 56 or both. It should be noted that these holes 52 can be of any shape.

The treatment or sealing section (54) can be deposited or adhered onto and envelope one or both sides of the final fabric 44 to facilitate the waterproof and breathability properties of the fabric 44. This section keeps liquid water from passing through the section but allows water vapor and other gases to move through the fabric section freely. The optional decorative or protective section 56 also can be added to one or both sides of the fabric 44 to change external properties of the final fabric such as texture, durability, or moisture wickability. As with the fabric embodiments in FIGS. 1 and 2, the sections can have different embodiments on the same plane. For instance, a section of the charge or recharge section 50 can use photovoltaics while another section can use piezoelectrics, or a section of the energy release plane can produce light while another section can produce heat. Similarly, one section of the plane can produce light while another section on the same plane can use photovoltaics to recharge the energy storage section. The sections also can be arranged in any order, including coplanar arrangements as well as stacking arrangements, and there can be multiple instances of each section in the final fabric.

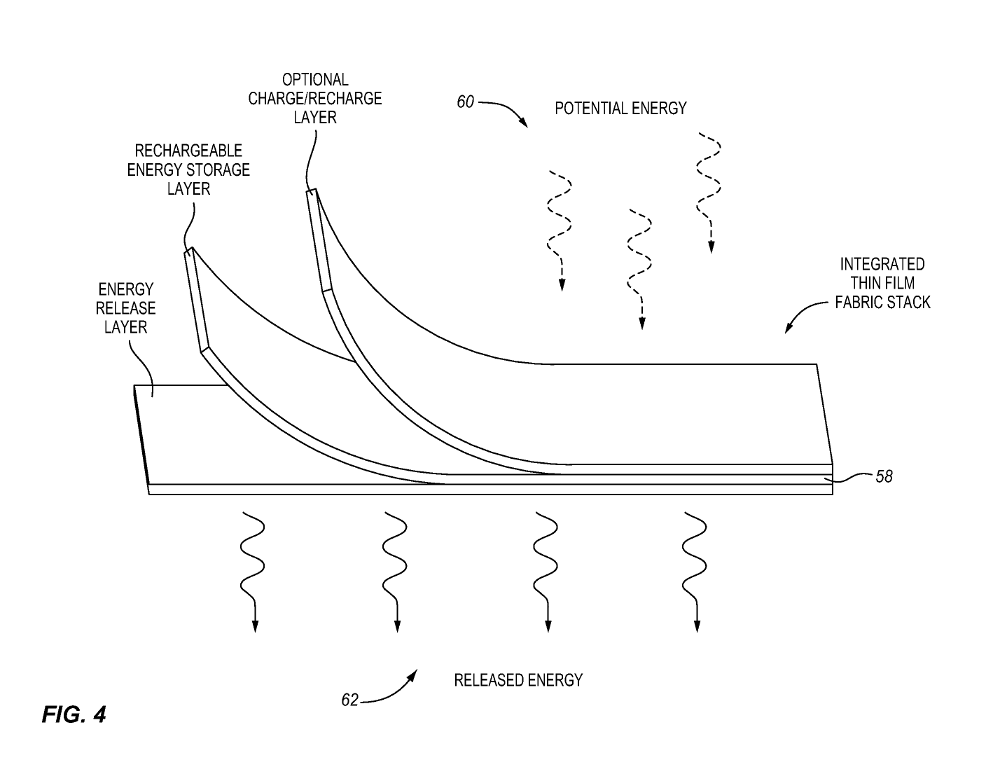

FIG. 4 illustrates a flexible, integrated fabric 58 capable of receiving surrounding energy 60 from many possible sources, converting it to electrical energy and storing it integral to the fabric, and then releasing the electrical energy in different ways 62.

### Self-Regulating Heated Wound Dressing Manufacturing

One method of manufacturing the individual sections into a custom, energized textile panel would consist of: 1) locating the energy storage, energy release, and possibly energy recharge sections adjacent to or on top of one another (depending on panel layout and functionality); 2) electrically interconnecting the various sections by affixing thin, flexible circuits to them that would provide the desired functionality; and then 3) laminating this entire system of electrically integrated sections between breathable, waterproof films. The preferred materials in the heating embodiment of a panel would consist of lithium polymer for the energy storage section, Positive Temperature Coefficient heaters for the energy release section, piezoelectric film for the recharge section, copper traces deposited on a polyester substrate for the thin, flexible electrical interconnects, and a high Moisture Vapor Transmission Rate polyurethane film as the encapsulating film or protective section. While cloth material can be used, preferably it would be laminated over the encapsulant film. The cloth could be any type of material and would correspond to the decorative section as described herein. The type of cloth would completely depend on the desired color, texture, wickability, and other characteristics of the exterior of the panel.

### Charge and Recharge Layers

There are many currently available options for the charge and recharge section in its several embodiments. In the case that the embodiment is using light energy to charge or recharge the energy storage section, flexible photovoltaic cells can be used. In the case that the embodiment is using fabric flexure and piezoelectric materials to generate electricity for storage in the energy storage section, films that are easily laminated and electrically integrated into the final fabric can be used. In the case that the embodiment is using an inductive or wireless charging system to produce electrical energy for storage, it can be laminated and electrically integrated into the final fabric.

Wireless energy transfer or wireless power transmission is the process that takes place in any system where electrical energy is transmitted from a power source to an electrical load without interconnecting wires. Wireless transmission is useful in cases where instantaneous or continuous energy transfer is needed but interconnecting wires are inconvenient, hazardous, or impossible. There are a number of wireless transmission techniques, and the following description characterizes several for the purpose of illustrating the concept.

Inductive charging uses the electromagnetic field to transfer energy between two objects. A charging station sends energy through inductive coupling to an electrical device, which stores the energy in the batteries. Because there is a small gap between the two coils, inductive charging is one kind of short-distance wireless energy transfer. When resonant coupling is used, the transmitter and receiver inductors are tuned to a mutual frequency, and the drive current can be modified from a sinusoidal to a non-sinusoidal transient waveform. This has an added benefit in that it can be used to “key” specific devices which need charging to specific charging devices to insure proper matching of charging and charged devices.

Induction chargers typically use an induction coil to create an alternating electromagnetic field from within a charging base station, and a second induction coil in the portable device takes power from the electromagnetic field and converts it back into electrical current to charge the battery. The two induction coils in proximity combine to form an electrical transformer.

The “electrostatic induction effect” or “capacitive coupling” is an electric field gradient or differential capacitance between two elevated electrodes over a conducting ground plane for wireless energy transmission involving high frequency alternating current potential differences transmitted between two plates or nodes. The electrostatic forces through natural media across a conductor situated in the changing magnetic flux can transfer energy to a receiving device.

The other kind of charging, direct wired contact (also known as conductive charging or direct coupling), requires direct electrical contact between the batteries and the charger. Conductive charging is achieved by connecting a device to a power source with plug-in wires, such as a docking station, or by moving batteries from a device to a charger.

It should also be noted that, in the case of a thermoelectric (Peltier) or photoelectric (photovoltaic) section that is used as an energy release embodiment, this section also can be used in a reversible fashion as an energy recharging section for the energy storage section(s). For example, if a system is producing a large amount of excess heat energy, say in the case of a garment used during high aerobic activity, that heat energy can be converted by the thermoelectric section to electricity for storage in the energy storage section(s) and can then be used reversibly back through a thermoelectric section for heating when there is an absence of heat after the aerobic activity has stopped. The same sort of energy harvesting technique could be used by the photoelectric (photovoltaic) sections to produce light when there's an absence of it and also to transform the light energy to electrical energy for storage in the energy storage sections when there is an excess of it. In the case of the piezoelectric embodiment, electrical energy can be created and stored during flexing and then used reversibly to stiffen the piezoelectric section if a stiffening of the fabric is required.

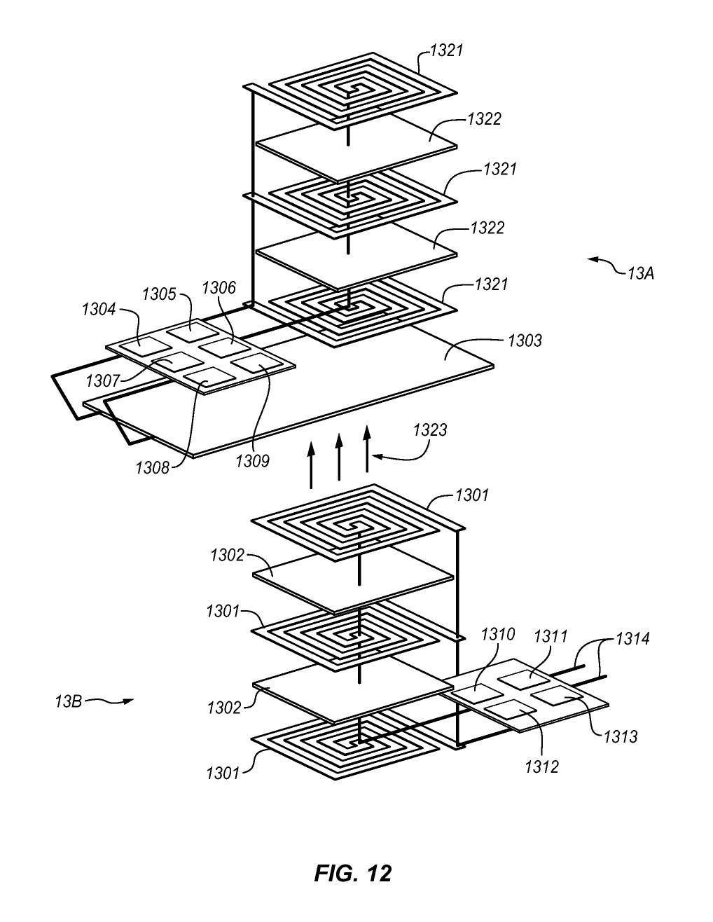

FIG. 12 illustrates a typical wireless apparatus for the transfer of energy into and out of the Self-Regulating Heated Wound Dressing. Printed circuit flexible heaters are constructed using several elements including Positive-Temperature-Coefficient (PTC) materials for delivering heat. Such constructions can be designed to operate in a steady-state or limiting modes. In the latter mode, the final temperature is bounded by the limiting resistance of the PTC material. Temperatures up to 80° C. can be achieved by allowing the heater to draw a small amount of current at a fixed potential. At the start of the heating, the current draw is typically a few microamperes; but as the heater approaches equilibrium, the current requirement is diminished to a level that is necessary to maintain the limiting temperature.

Critical parameters for heater construction include physical and chemical characteristics of the electrodes and the applied voltage. PTC material can be deposited using standard screen-printing techniques in a wide range of thicknesses. As the deposit thickness increases, its resistance decreases and the observed temperature decreases. Electrode spacing as small as 250 microns (0.010″) can be achieved. Typical spacings are in the range of 0.75 mm to 1.5 mm. Heating temperatures at a fixed potential increase as the electrode spacing decreases. The temperature response as function of applied potential is always positive. Applied voltages are usually in the range of 3 VDC to 12 VDC.

As shown in FIG. 12, the wireless power receiver 13A and wireless power transmitter 13B are each constructed from multiple layers of Flexible Printed Circuit (FPC) coils 1321 and 1301, respectively, which are each separated by magnetic cores 1322 and 1302, respectively, (preferably soft magnetic cores). These magnetic cores 1322, 1302 function to increase the field strength (range/power). A battery 1303 stores the electrical energy in the wireless power receiver 13A. A voltage conversion circuit interfaces the FPC coils 1321 with the battery 1303 (which can be the energy storage section 14) and comprises a voltage regulator 1304, resonance capacitor 1305, tuning circuit 1306, and charging/protection circuit 1307 which operate in well-known fashion to output a controlled voltage at port 1308 once the presence of a wireless charging transmitter is detected by the charging pad sense circuit 1309. In the wireless power transmitter 13B, a resonant circuit, which includes resonance capacitor 1310, signal conditioning circuit 1311, and tuning circuit 1312, operates to output an energy field 1323 to wireless power receiver 13A. In response to chargeable device sense circuit 1313 detecting the presence of a wireless power receiver 13A (such as the energy recharge section 18), the wireless power transmitter 13B converts the power received from power main 1314 to a wireless signal 1323 output via FPC coils 1301 to the wireless power receiver 13A (such as the energy recharge section 18).

### Protective Layers

There are many available products that can be used for the protective and decorative and bandage section(s) that are engineered for next-to-skin wickability, fibrous, fleece-type comfort, water repellency, specific color, specific texture, and many other characteristics that can be incorporated by laminating that section into the final fabric. In addition, the bandage fabric can be impregnated with therapeutic materials, such as medications, including thermally activated medications. There are also many ThermoPlastic Urethanes (TPUs) available for use as sealing and protective envelopes. These materials exhibit very high Moisture Vapor Transmission Ratios (MVTRs) and are extremely waterproof, allowing the assembled energy storage, release, and recharge sections to be enveloped in a highly breathable, waterproof material that also provides a high degree of protection and durability. In addition to the TPUs, which are a solid monolithic structure, there are also microporous materials that are available for use as breathable, waterproof sealing and protective envelopes. This microporous technology is commonly found in Gore products and can also be used in conjunction with TPUs. It should also be noted that, when laminating these breathable waterproof envelopes around the assembled sections, care must be taken, whether one is using an adhesive or not, to maintain the breathability of the laminate. If adhesive is being used, this adhesive must also have breathable characteristics. The same should be said for a laminate process that does not use adhesive. Whatever the adhesion process is, it needs to maintain the breathability and waterproofness of the enveloping protective section, providing these are traits deemed necessary for the final textile panel.

As an optional treatment or sealing, section 40 can be deposited on one or both sides of the final fabric 28 to facilitate the waterproof and breathability properties of the fabric. This enveloping section keeps liquid water from passing through but allows water vapor and other gases to move through it freely. An optional protective or decorative section 42 also can be added to change external properties of the final fabric such as texture, durability, stretchability, or moisture wickability.

### Embedding Electronic Components in Film Substrates Summary

The present Self-Regulating Heated Wound Dressing also provides techniques for sealing devices, such as electronic circuits, components, and electrical energy storage devices, inside a highly flexible, robust laminate panel for subsequent integration into a larger system. This Self-Regulating Heated Wound Dressing provides a system where the devices, such as electronic circuits, components, and energy storage devices, are embedded between laminated film substrates to form a flexible, environmentally sealed, finished laminate able to be integrated into a larger system such as a garment or accessory. The embedded circuits, components, and energy storage devices can be included in many different substrate layers within the finished laminate. The devices also can be located in separate panels and connected together via external connectors to provide a larger system. It is possible to produce a finished laminate with environmentally sealed, embedded electrical components, circuits, and energy storage devices that is thin and flexible.

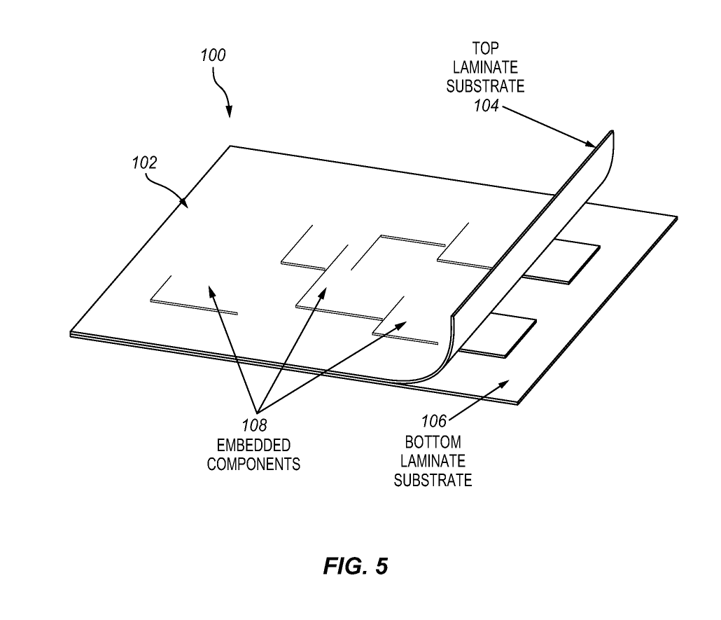

FIG. 5 shows a segment 100 of laminate material 102 having a top laminate layer 104 and a bottom laminate layer 106. Embedded between these two layers 104, 106 are devices 108, such as electrical circuits, electrical energy storage devices, electromagnetic devices, semiconductor chips, heating or cooling elements, or both, light emission devices such as incandescent lights or LEDs or both, sensors, speakers, RF transceivers, antennae, and the like.

### Battened Adhesive Lamination Background

Currently, there are many substrate or layer adhesion systems that consist of solid or patterned adhesive applied to film for the purpose of affixing the film to another object. However, there is not an adhesion system coupled with a lamination manufacturing technique for producing a single laminate that maximizes adhesive strength between the films, maximizes the MVTR properties of the finished laminate, and maintains a robust fluid barrier for the electronic components embedded between its films.

The present Self-Regulating Heated Wound Dressing provides a lamination system and technique that maximizes substrate film adhesion strength and maintains a robust fluid barrier for embedded electronic components while also maximizing MVTR through the finished laminate. By using striped adhesion on the substrate layers and orienting the layers during lamination so that the adhesive strips are at an angle other than parallel to one another, the present Self-Regulating Heated Wound Dressing creates a finished single laminate that is strong, highly breathable, and retains a sectioned fluid barrier so embedded components are protected if the finished laminate is somehow compromised. This adhesion technique can be used with many layers of substrates to create a final laminate with many battened adhesive layers. The adhesion also can consist of a single or multiple patterned adhesive layers, as long as the resultant adhesive pattern when laminated forms a closed adhesive batten.

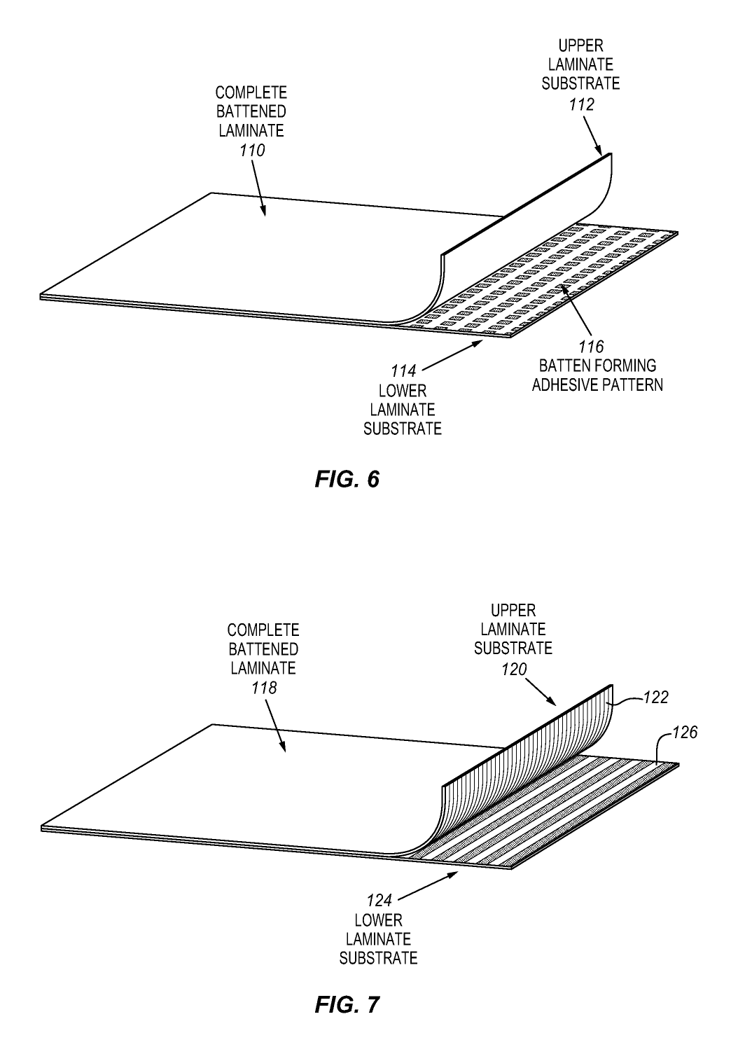

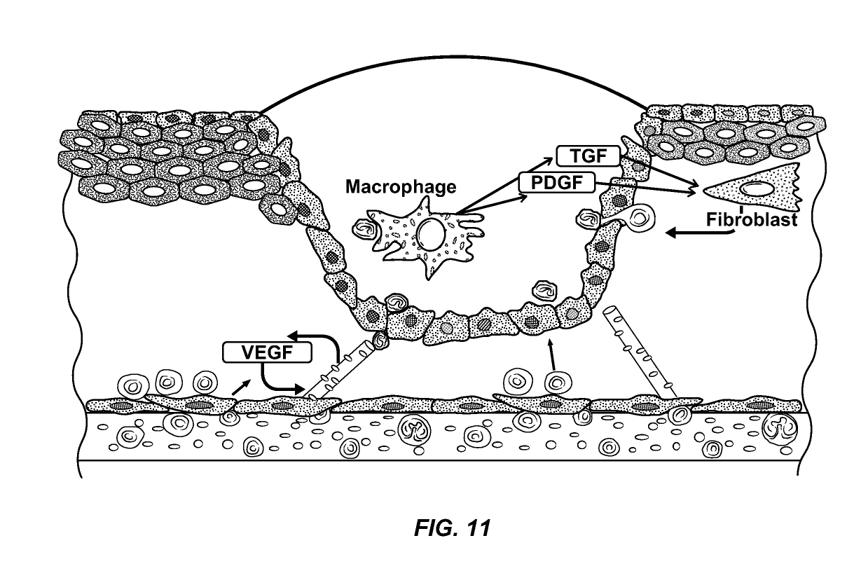

FIG. 6 shows a battened laminate section 110 with upper and lower substrates 112, 114, respectively, that are adhered together by a batten-forming adhesive pattern 116 that is shown on the lower laminate substrate 114. FIG. 11 shows a complete battened laminate section 118 in which an upper laminate substrate 120 has longitudinal strips of adhesive 122 and the lower laminate substrate 124 has transverse strips of adhesive 126. When these substrates 120, 124 are pressed together, the adhesive strips 122, 126 form a batten checkerboard pattern.

### Energized Textile Lamination Press Summary

While there are currently systems that can be used for the lamination of thin, flexible substrates around electronic circuits and components, there is no system capable of allowing an operator to place electronic circuits and components at registration points imparted to the film substrate and then initiate a lamination of the two films around the placed circuits and components to ensure no air bubbles are formed between the lamination films. The present Self-Regulating Heated Wound Dressing provides a lamination system that allows the user to place devices, such as circuits and components, in a specific geometry between two film sections, panels, layers, or substrates while ensuring that no unwanted air is trapped between the laminations as the lamination occurs. The registration points can be transmitted to the substrate via light or via a physical jig that allows the embedded devices to be placed and held as the lamination process occurs.

To ensure that air bubbles are not trapped between the substrates or sections as the lamination process occurs, the contact surface of the press incorporates a curved or domed convex deformable surface that presses air out from a single location towards the current unsealed areas while not damaging components in the current laminated areas as the entire surface receives the pressure and possibly radiant energy required to continuously laminate the panel. The introduction of energized textile panels creates the need for specific manufacturing techniques and processes that enable energized fabric panels to be mass produced with a high degree of quality.

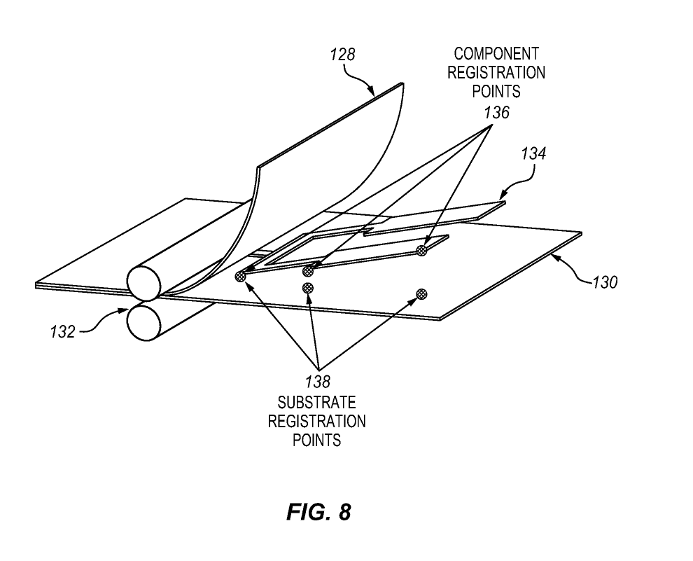

FIG. 8 illustrates one embodiment of the present disclosure in which upper and lower layers 128, 130, respectively, are compressed together between a pair of rollers 132. It is to be understood that a single roller pressing on a support surface could also be used. An electric component 134 is placed between the two layers 128, 130 and positioned by component registration points 136 and substrate registration points 138 as described above.

### Energy Storage Layer

A thin film, lithium ion polymer battery is an ideal flexible thin, rechargeable, electrical energy storage section. These batteries consist of a thin film anode layer, cathode layer, and electrolytic layer; and each battery forms a thin, flexible sheet that stores and releases electrical energy and is rechargeable. Carbon nanotubes can be used in conjunction with the lithium polymer battery technology to increase capacity and would be integrated into the final fabric in the same manner as would a standard polymer battery. It should be noted that the energy storage section should consist of a material whose properties do not degrade with use and flexing. In the case of lithium polymers, this generally means the more the electrolyte is plasticized, the less the degradation of the cell that occurs with flexing.

Another technology that can be used for the energy storage section is a supercapacitor or ultracapacitor which uses different technologies to achieve a thin, flexible, rechargeable energy storage film and are good examples in the ultra- and super-capacitor industry as to what is currently available commercially for integration and use in this Self-Regulating Heated Wound Dressing.

Thin film micro fuel cells of different types (PEM, DFMC, solid oxide, MEMS, and hydrogen) can be laminated into the final fabric to provide an integrated power source to work in conjunction with (hybridized), or in place of, a thin film battery or thin film capacitor storage section.

### Energy Release Layer

In the energy release section, there are several embodiments including, but not limited to, heating, cooling, light emission, and energy transmission. For the heating embodiment, a normal thin wire or etched thin film resistance heater works well. A Positive Temperature Coefficient resistive heater also works very well for a thin film, self-regulating, heater section. In the case of the Positive Temperature Coefficient resistive heater, its heater is built to regulate itself specifically to a temperature determined before manufacture, which effect is termed “constant thermal emission” or “constant thermal output” herein. This means that the resistive heating element changes its resistance depending on the instantaneous temperature of the heater without the use of sensors and added circuitry. In addition, the Positive Temperature Coefficient resistive heater is powered by the DC voltage output by the energy storage layer without the need for voltage converters or complex control circuitry.

Viewing heating and cooling more expansively, the thermoelectric effect is the direct conversion of temperature differences to electric voltage and vice versa. A thermoelectric device creates a voltage when there is a different temperature on each side. Conversely, when a voltage is applied to it, it creates a temperature difference (known as the Peltier effect). At atomic scale (specifically, charge carriers), an applied temperature gradient causes charged carriers in the material, whether they are electrons or electron holes, to diffuse from the hot side to the cold side, similar to a classical gas that expands when heated; hence, the thermally induced current. Mobile charged carriers migrating to the cold side leave behind their oppositely charged and immobile nuclei at the hot side, thus giving rise to a thermoelectric voltage (“thermoelectric” refers to the fact that the voltage is created by a temperature difference). Since a separation of charges also creates an electric potential, the buildup of charged carriers onto the cold side eventually ceases at some maximum value since there exists an equal amount of charged carriers drifting back to the hot side as a result of the electric field at equilibrium. Only an increase in the temperature difference can resume a buildup of more charge carriers on the cold side and, thus, lead to an increase in the thermoelectric voltage. Incidentally, the thermopower also measures the entropy per charge carrier in the material. To be more specific, the partial molar electronic heat capacity is said to equal the absolute thermoelectric power multiplied by the negative of Faraday's constant.

This Peltier effect can be used to generate electricity, to measure temperature, to cool objects, or to heat them or cook them. Because the direction of heating and cooling is determined by the polarity of the applied voltage, thermoelectric devices make very convenient temperature controllers. Traditionally, the term “thermoelectric effect” or “thermoelectricity” encompasses three separately identified effects: the Seebeck effect, the Peltier effect, and the Thomson effect.

The Seebeck effect is the conversion of temperature differences directly into electricity.

The effect is that a voltage, the thermoelectric EMF, is created in the presence of a temperature difference between two different metals or semiconductors. This causes a continuous current in the conductors if they form a complete loop. The voltage created is of the order of several microvolts per Kelvin difference. One such combination, copper-constantan, has a Seebeck coefficient of 41 microvolts per Kelvin at room temperature. The thermopower, thermoelectric power, or Seebeck coefficient of a material measures the magnitude of an induced thermoelectric voltage in response to a temperature difference across that material. The term “thermopower” is a misnomer, since it measures the voltage or electric field induced in response to a temperature difference, not the electric power.

Refrigeration is the process of removing heat from an enclosed space, or from a substance, and moving it to a place where it is unobjectionable. The primary purpose of refrigeration is lowering the temperature of the enclosed space or substance and then maintaining that lower temperature. The term “cooling” refers generally to any natural or artificial process by which heat is dissipated. The process of artificially producing extreme cold temperatures is referred to as “cryogenics.” Cold is the absence of heat; hence, in order to decrease a temperature, one “removes heat” rather than “adding cold.” In order to satisfy the Second Law of Thermodynamics, some form of work must be performed to accomplish this. The work traditionally is done by mechanical work but can also be done by magnetism, laser, or other means.

Thermoelectric cooling uses the Peltier effect to create a heat flux between the junction of two different types of materials. The thermoelectric effect is the direct conversion of temperature differences to electric voltage and vice versa. A thermoelectric device creates a voltage when there is a different temperature on each side. Conversely, when a voltage is applied to it, it creates a temperature difference (known as the Peltier effect). At atomic scale (specifically, charge carriers), an applied temperature gradient causes charged carriers in the material, whether they are electrons or electron holes, to diffuse from the hot side to the cold side, similar to a classical gas that expands when heated; hence, the thermally-induced current.

All of these heating elements are deposited on a thin flexible substrate, usually kapton or polyester, which then can be laminated with or without an adhesive to the other fabric sections, or the heating elements can be directly deposited on an adjoining fabric section. For instance, the heater element can be deposited directly on the packaging layer of a lithium polymer battery and then covered with a thin film of polyester, kapton, urethane, or some other thin flexible material to encapsulate and insulate the heating element and/or fabric section.

For the cooling embodiment of the energy release section, a thin film, superlattice, thermoelectric cooling device is ideal for integration into the final fabric. Being a thin film device, it can be deposited using another of the fabric sections as its substrate or it can be deposited on a separate substrate and then laminated with or without an adhesive to the other existing fabric sections.

### Wound Healing Biology

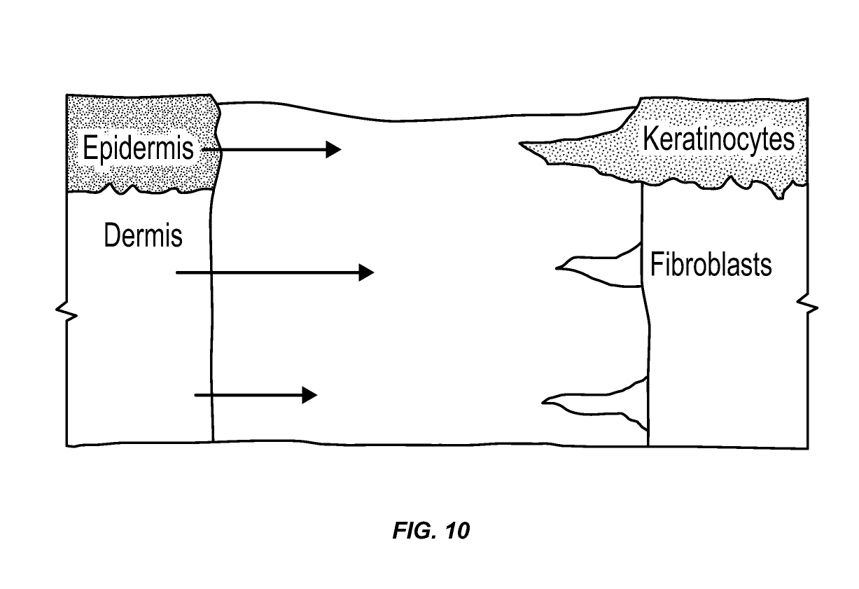

FIG. 10 illustrates a typical subcutaneous wound and the initial stages of the wound healing process at the skin or surface layer of a living organism, and FIG. 11 illustrates a typical subcutaneous wound and the various biological reactions involved in wound healing. In particular, when the epidermis and dermis are compromised, the wound edges are separated by a void, which fills with blood, which clots to form a fibrin clot to prevent the incursion of hostile agents, such as bacteria. The epidermis then begins to produce Keratinocytes and the dermis produces fibroblasts to begin to grow the epidermis and dermis, respectively, into the void filled by the blood clot. Thus, the repair process incorporates regrowth of the damaged tissue toward the opposite edges of the wound to recreate the original epidermis and dermis tissue.

In FIG. 11, additional detail is provided to further illustrate this process. In particular, the presence of macrophages is illustrated, where the macrophages attack, encapsulate and remove foreign bodies, such as necrotic cellular debris, from the wound site. When a leukocyte enters damaged tissue through the endothelium of a blood vessel (a process known as the “leukocyte extravasation”), it undergoes a series of changes to become a macrophage. Monocytes are attracted to a damaged site by chemical substances through chemotaxis, triggered by a range of stimuli including damaged cells, pathogens, and cytokines released by macrophages already at the site.

Neutrophil granulocytes are generally referred to as “neutrophils”, are the most abundant type of white blood cells in mammals, and form an essential part of the innate immune system. Being highly motile, neutrophils quickly congregate at a focus of infection, attracted by cytokines expressed by activated endothelium, mast cells, and macrophages. Neutrophils express and release cytokines, which in turn amplify inflammatory reactions by several other cell types. In addition to recruiting and activating other cells of the immune system, neutrophils play a key role in the front-line defense against invading pathogens.

In addition, fibroblasts are present and consist of a type of cell that synthesizes the extracellular matrix and collagen, which is the structural framework (stroma) for animal tissues, and plays a critical role in wound healing. Fibroblasts are the most common cells of connective tissue in animals.

An integral component of all of the above defense mechanisms is the presence of blood vessels to provide the delivery mechanism for the macrophages and neutrophils to the wound site and the removal of waste products from the wound site. The stimulation of the circulatory system at the wound site can be accomplished by a number of mechanisms, and the external application of heat at the wound site is a preferable manner to non-invasively and controllably increase circulation. A traditional problem with the application of heat to a wound is the thermal cycling of heated bandages, where the initial temperature of the bandage is above the desired temperature and the thermal output rapidly diminishes to a level below that which produces the desired result, quickly reducing the efficacy of the heated bandage. The frequent replacement of the heated bandage can be damaging to the healing process and costly in terms of materials and staff time required to manage the process.

### Chronic Venous Ulcers (CWF) Example

Wound fluid from Chronic Venous Ulcers (CWF) has been shown to inhibit cellular proliferation, contributing to the impaired healing of chronic ulcers. CWF has been shown to specifically inhibit proliferation of dermal fibroblast and endothelial cells, thus retarding the healing process. CWF inhibits the proliferation of newborn dermal fibroblasts, inhibits DNA synthesis in human neonatal fibroblasts, and arrests cells in the G1 phase of the cell cycle. A recent report has suggested that CWF-induced suppression of growth involves modulation of cell cycle-dependent proteins, in particular: pRb, cyclin D1, CDK4, and p21Cip1/Waf1.1.

This growth inhibitory activity was shown to be heat sensitive in that, when CWF was heated, there was a temperature-dependent reduction in the growth inhibitory activity. Heat-sensitivity of growth inhibitory activity in CWF suggests that a thermal wound therapy that warms the wound fluid may be beneficial in treating leg ulcers. Warming of wound fluid in chronic leg ulcers would counteract the growth inhibitory activity of CWF, allowing normal cellular proliferation in the wound. Thus, a noncontact thermal wound therapy can counteract growth inhibitory activity in CWF. As an example, heating CWF in vitro with a thermal wound therapy allowed normal proliferation and morphology of dermal fibroblasts. The maximal temperature of CWF reached by heating CWF with noncontact thermal wound therapy for 72 hours was 35° C., a temperature well below the normal body temperature of 37° C. Warming of CWF using noncontact thermal wound therapy blocks the CWF-induced suppression of Rb phosphorylation. This is achieved, in part, by sustaining the level of cyclin D1/CDK4 complex that phosphorylates Rb. In addition, warming CWF also blocked CWF-induced increases in the growth inhibitory protein p21Cip1/Waf1. Because p21Cip1/Waf1 prevents cyclin Dl/CDK4 complex-mediated phosphorylation of Rb, a decreased level of p21Cip1/Waf1 in cells treated with heated CWF would result in the normal level of pRb, thus allowing proper progression of cells through G1 into S phase during proliferation of dermal fibroblasts. Therefore, a noncontact thermal therapy prevents CWF-induced inhibition of the growth of dermal fibroblasts, resulting in enhanced wound healing. Moreover, the enhanced healing by a thermal wound therapy is not due to a general nonspecific stimulation of fibroblast growth, but it is mediated through specific positive modulations on the levels of cell cycle-regulatory proteins. The maintenance of critical cell cycle-regulatory proteins, such as pRb and cyclin D1/CDK4 complex, may be critical for the proper regeneration and healing of the wounds.

### Self-Regulating Heated Wound Dressings

A traditional problem with the application of heat to a wound site is the thermal cycling of heated bandages, where the initial temperature of the bandage is above the desired temperature, and the thermal output rapidly diminishes below the desired level, quickly reducing the efficacy of the heated bandage. The frequent replacement of the heated bandage can be damaging to the healing process and costly in terms of materials and staff time required to manage the process.

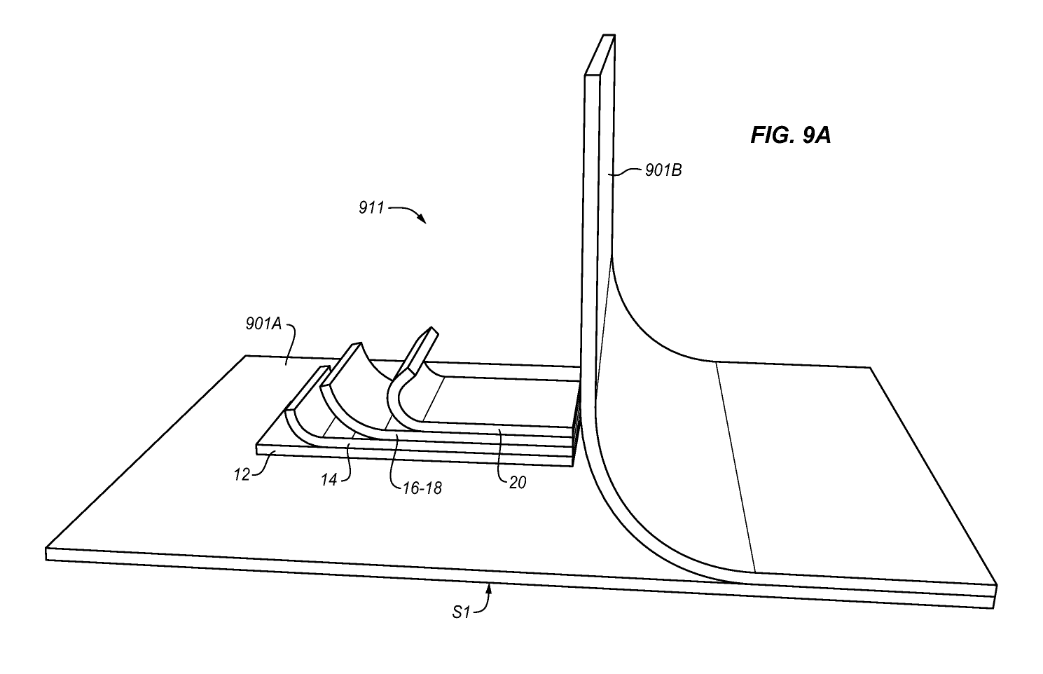

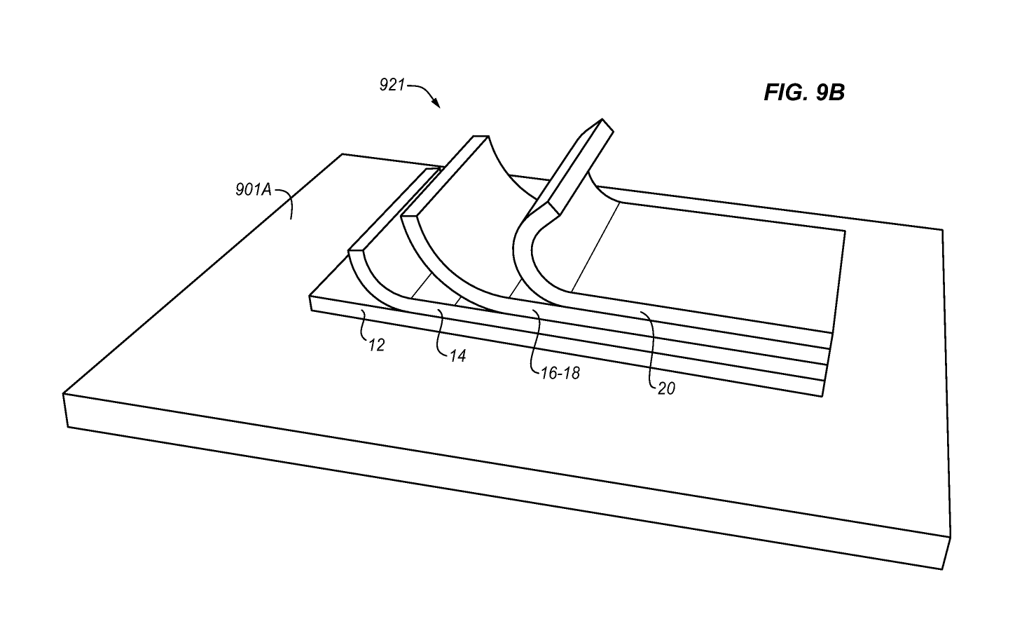

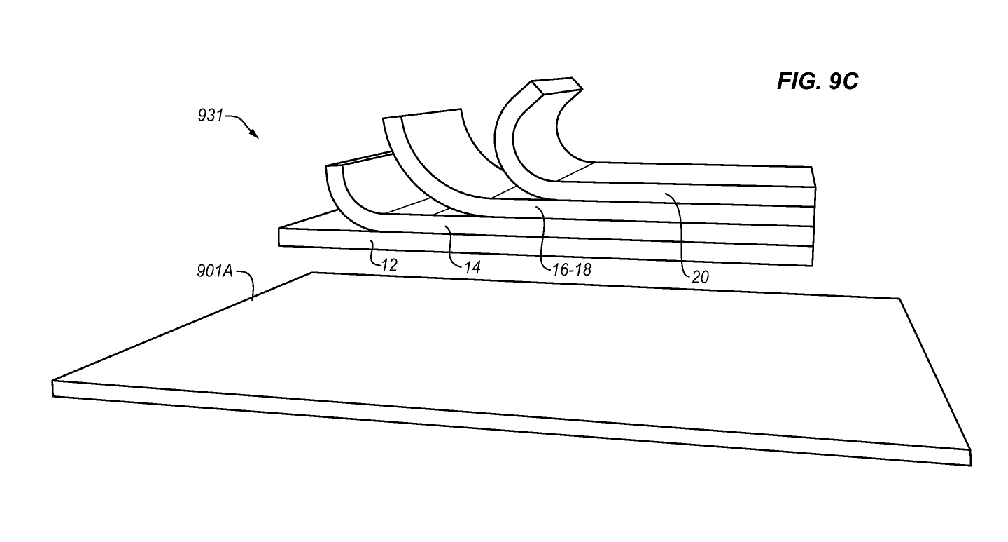

FIGS. 9A-9C illustrate typical heated wound bandages using the Self-Regulating Heated Wound Dressing, which provides a predetermined thermal output to maintain a substantially constant temperature at the wound site for an extended period of time. The Self-Regulating Heated Wound Dressing is non-invasive, self-contained to enable the patient to be ambulatory, and also is wirelessly rechargeable to provide the capability for producing a constant thermal output over an extended period of time without having to remove the dressing. The heated wound dressing can be coupled with an absorbent bandage fabric to interface between the wound surface and the Self-Regulating Heated Wound Dressing. In addition, the bandage fabric can be impregnated with therapeutic materials, such as medications, including thermally activated medications. Dressing attachment apparatus (not shown) can also be provided, such as adhesive strips, Velcro strips, adhesive wraps, and the like, to enable the efficient positioning and fixation of the Self-Regulating Heated Wound Dressing to the wound site.

FIG. 9A illustrates a “pocket” type of wound bandage 911 where the bandage portion 901 of the Self-Regulating Heated Wound Dressing comprises two layers 901A, 901B of bandage or a bandage layer 901A with a cover layer 901B, which enclose the active elements 12-18 of the Self-Regulating Heated Wound Dressing 911. One side S1 of the Self-Regulating Heated Wound Dressing 911 is placed in contact with the surface to be treated and can be infused with medicines or other materials to provide enhanced treatment of the surface and underlying tissue. The heating element 12 of the Self-Regulating Heated Wound Dressing 911 generates a constant temperature output which is transmitted through the contact layer 901A to the surface being treated. The Self-Regulating Heated Wound Dressing 911 can be held in place by the use of a wrap or tape applied over the Self-Regulating Heated Wound Dressing 911, or can include adhesive strips which can be deployed and used to secure the Self-Regulating Heated Wound Dressing 911 in place.

FIG. 9B illustrates an adhesively attached bandage layer version of the Self-Regulating Heated Wound Dressing 921, where a single layer of bandage material 901A is adhesively affixed to one side of the laminated collection of active elements. As above, the bandage 901A can be infused with medicines or other materials to provide enhanced treatment of the surface and underlying tissue. The Self-Regulating Heated Wound Dressing 921 can be held in place by the use of a wrap or tape applied over the Self-Regulating Heated Wound Dressing 921, or can include adhesive strips which can be deployed and used to secure the Self-Regulating Heated Wound Dressing 921 in place.

FIG. 9C illustrates the instance of using a bandage 901A which is not an integral part of the physical structure of the Self-Regulating Heated Wound Dressing 931 but is external to the laminated collection of active elements. The bandage 901A can be infused with medicines or other materials to provide enhanced treatment of the surface and underlying tissue.

The laminated collection of active elements 12-20 can be either the porous structure described above to facilitate air circulation to the wound area or can be an impervious structure to prevent fluid infusion into the laminated collection 12-20 of active elements, where the laminated collection 12-20 of active elements can be sterilized for repetitive use. The laminated collection 12-20 of active elements can also be implemented in various other forms, such as a cap for use on a subject's head to provide warming of the scalp, or a large “wrap around” structure to encircle a subject's limb. There are numerous other configurations that are possible and well-known in the art but are not described herein for the sake of brevity. Also, while the use for medical treatment has been described, the use for cosmetic purposes, topical heat treatment for muscle pain, etc. also are included in this architecture. The fundamental concepts taught by this description are reflected in the language of the claims that are appended hereto, and an expansive interpretation of the structure recited therein is supported by the above description.

### SUMMARY

The Self-Regulating Heated Wound Dressing includes an energy storage section adapted to store electrical energy; an energy release section coupled to the energy storage section and configured to receive electrical energy from the energy storage section and to utilize the electrical energy in the generation of a thermal energy used to self-regulate the temperature of a heated wound dressing; and an energy recharge section, coupled to the energy storage section, adapted to receive or collect energy and convert the received or collected energy to electrical energy either for storage by the energy storage section or for use by the energy release section or simultaneous storage in the energy storage section and immediate use by the energy release section.

What is claimed as new and desired to be protected by Letters Patent of the United States is:

1. A Self-Regulating Heated Wound Dressing for the generation of thermal energy, comprising:
an energy storage section configured to store electrical energy;
an energy release section configured to generate a substantially constant thermal emission by utilizing the electrical energy stored in the energy storage section;
an energy recharge section adapted to collect energy from a source located external to said material and convert the collected energy to electrical energy for storage by the energy storage section, for immediate use by the energy release section, or simultaneous storage in the energy storage section and use by the energy release section;
a bandage section in thermal communication with the energy release section for providing a surface for contact with a site on a subject to enable the controllable transfer of heat from the energy release section to the site; and
a control process for regulating at least one of energy storage and energy release in the energy storage and energy release sections, respectively; and
wherein the energy storage and said energy recharge sections are encapsulated in a laminate to form a sheet-like material.

2. The Self-Regulating Heated Wound Dressing for the generation of thermal energy of claim 1 wherein the energy storage and energy release sections comprise first and second layers, respectively, and are arranged in at least one of: coplanar arrangements, layers, planes, and other stacking arrangements, and there can be multiple instances of each section.

3. The Self-Regulating Heated Wound Dressing for the generation of thermal energy of claim 1 wherein said energy recharge section is coupled to at least the energy storage section and formed with the energy storage and energy release sections in the laminate.

4. The Self-Regulating Heated Wound Dressing for the generation of thermal energy of claim 1 wherein said energy recharge section comprises:
a wireless energy transfer circuit for receiving electric power from a source located external to said Self-Regulating Heated Wound Dressing via a one of: inductive and wireless charging.

5. The Self-Regulating Heated Wound Dressing for the generation of thermal energy of claim 1 wherein the energy storage, energy release, and energy recharge sections comprise first, second, and third layers, respectively, and are arranged in one of: coplanar arrangements, layers, planes, and other stacking arrangements, and there can be multiple instances of each section.

6. The Self-Regulating Heated Wound Dressing for the generation of thermal energy of claim 1 wherein the energy storage and energy release sections are formed to be flexible and to have at least one of the following characteristics of breathability, moisture wickability, water resistance, waterproof, and stretchability.

7. The Self-Regulating Heated Wound Dressing for the generation of thermal energy of claim 1 wherein the energy release section comprises:
a self-regulating heat generator for maintaining a substantially constant temperature absent the use of control circuitry.

8. The Self-Regulating Heated Wound Dressing for the generation of thermal energy of claim 7 wherein the self-regulating heat generator comprises:
a Positive Temperature Coefficient resistive heater where the resistive heating element changes its resistance depending on the instantaneous temperature of the heater without the use of sensors and added circuitry.

9. The Self-Regulating Heated Wound Dressing for the generation of thermal energy of claim 1 wherein the bandage section comprises:
bandage material adhesively affixed to a surface of the laminate material.

10. The Self-Regulating Heated Wound Dressing for the generation of thermal energy of claim 1 wherein the bandage section comprises:
bandage material for enclosing the laminate material.

11. The Self-Regulating Heated Wound Dressing for the generation of thermal energy of claim 1 wherein the bandage section comprises:
bandage material external to and in contact with a surface of the laminate material.

12. A Self-Regulating Heated Wound Dressing for the generation of thermal energy, comprising:
an energy storage section configured to store electrical energy;
an energy release section configured to generate thermal emissions by utilizing the electrical energy stored in the energy storage section; and
an energy recharge section adapted to collect energy from a source located external to said material and convert the collected energy to electrical energy for storage by the energy storage section, for immediate use by the energy release section, or simultaneous storage in the energy storage section and use by the energy release section;
wherein the energy storage, energy release, and energy recharge sections are encapsulated in a laminate to form a sheet-like material; and
a control process for regulating at least one of energy storage and energy release in the energy storage and energy release sections, respectively.

13. The Self-Regulating Heated Wound Dressing for the generation of thermal energy of claim 12 wherein the energy storage and energy release sections comprise energy storage and energy release layers, respectively, and are arranged in at least one of: coplanar arrangements, layers, planes, and other stacking arrangements, and there can be multiple instances of each section.

14. The Self-Regulating Heated Wound Dressing for the generation of thermal energy of claim 12 wherein said energy recharge section is coupled to at least the energy storage section and formed with the energy storage and energy release sections in the laminate.

15. The Self-Regulating Heated Wound Dressing for the generation of thermal energy of claim 12 wherein said energy recharge section comprises:
a wireless energy transfer circuit for receiving electric power from a source located external to said Self-Regulating Heated Wound Dressing via a one of: inductive and wireless charging.

16. The Self-Regulating Heated Wound Dressing for the generation of thermal energy of claim 12 wherein the energy storage, energy release, and energy recharge sections comprise first, second, and third layers, respectively, and are arranged in at least one of: coplanar arrangements, layers, planes, and other stacking arrangements, and there can be multiple instances of each section.

17. The Self-Regulating Heated Wound Dressing for the generation of thermal energy of claim 12 wherein the energy storage and energy release sections are formed to be flexible and to have at least one of the following characteristics of breathability, moisture wickability, water resistance, waterproof, and stretchability.

18. The Self-Regulating Heated Wound Dressing for the generation of thermal energy of claim 12 wherein the energy release section comprises:
a self-regulating heat generator for maintaining a substantially constant temperature absent the use of control circuitry.

19. The Self-Regulating Heated Wound Dressing for the generation of thermal energy of claim 18 wherein the self-regulating heat generator comprises:
a Positive Temperature Coefficient resistive heater where the resistive heating element changes its resistance depending on the instantaneous temperature of the heater without the use of sensors and added circuitry.

20. The Self-Regulating Heated Wound Dressing for the generation of thermal energy of claim 12 wherein the bandage section comprises:
bandage material adhesively affixed to a surface of the laminate material.

21. The Self-Regulating Heated Wound Dressing for the generation of thermal energy of claim 12 wherein the bandage section comprises:
bandage material for enclosing the laminate material.

22. The Self-Regulating Heated Wound Dressing for the generation of thermal energy of claim 12 wherein the bandage section comprises:
bandage material external to and in contact with a surface of the laminate material.
